(19) United States
(12) Patent Application Publication
(10) Pub. No.: US 2026/0262975 A1
(43) Pub. Date: Sep. 10, 2026

(54) SKIN TONE DETECTION FOR PHOTOPLETHYSMOGRAPHY CALIBRATION

(71) Applicant: GOOGLE LLC, Mountain View, CA (US)

(72) Inventor: Sumukh Shevde, Encinitas, CA (US)

(21) Appl. No.: 19/164,098

(22) PCT Filed: Apr. 26, 2023
(86) PCT No.: PCT/US2023/020027

§ 371 Date: Sep. 10, 2025

(51) Int. Cl. A61B 5/1495; A61B 5/00; A61B 5/1455

(52) U.S. Cl. CPC A61B 5/1495; A61B 5/002; A61B 5/14552; A61B 5/681; A61B 2560/0233; A61B 2562/043

(57) ABSTRACT

Techniques of calibrating a PPG sensor include accessing photos on a companion device connected to a wearable device having PPG sensors and deriving skin tone information from the photos. Based on the skin tone information, the wearable device performs a calibration operation to set a parameter value (power, wavelength) of the PPG sensor.

### TECHNICAL FIELD

This description relates in general to wearable devices and mobile devices, and in particular, to wearable devices including a photoplethysmography (PPG) sensor.

### BACKGROUND

This disclosure relates to photoplethysmography (PPG). PPG uses electromagnetic (EM) radiation (usually in the infrared (IR) band) to measure volumetric variations of blood circulation. A typical PPG device contains a source of EM radiation and a photodetector. The EM radiation source emits EM radiation to a tissue and the photodetector measures the reflected light from the tissue. The reflected light is proportional to blood volume variations. Many PPG sensors use an infrared light emitting diode (IR-LED) or a green LED as the main EM radiation source. IR-LEDs may be used for measuring the flow of blood that is more deeply concentrated in certain parts of body such as the muscles, whereas green light may be used for calculating the absorption of oxygen in oxyhemoglobin (oxygenated blood) and deoxyhemoglobin (blood without oxygen present).

### SUMMARY

The improvements discussed herein are directed to methods and systems for calibrating a PPG sensor of a wearable device using skin tone data obtained from images on a mobile device. Some PPG sensors may be found on wearable devices that are worn on a user's wrist, e.g., a smartwatch. In some cases, the measurements made by a PPG sensor are sensitive to the skin tone of the user. For example, darker skin can absorb more light from an LED source than lighter skin; the reflected signals returned from the same LED source under otherwise similar physiological conditions can be vastly different. Accordingly, the PPG sensor is calibrated so that the measurements made are as accurate as possible. In this disclosure, the skin tone of a user is determined, e.g., using images stored on a mobile device connected to the wearable device having the PPG sensor. For example, upon a first PPG measurement from a user, processing circuitry of the wearable device determines that there is a connection between the wearable device and a mobile device belonging to the user. Once the determination is made, the wearable device sends the mobile device a prompt to select at least one image (photo) of the user. Each photo, in some implementations, has metadata that indicates a value for the skin tone of the user. In one example, the value of the skin tone is a number between 1 and 6 inclusive (e.g., the Fitzpatrick Skin Phototype Classification). In another example, the value of the skin tone is an alphanumeric identifier. The mobile device sends the skin tone value to the wearable device; in some implementations, the mobile device sends an image to the wearable device, from which the skin tone value may be obtained. The processing circuitry of the wearable device then sets a parameter of the PPG sensor measurement based on the skin tone value or performs a calibration operation on the PPG sensor based on the skin tone value. In one example, the processing circuitry of the wearable device causes an optical power of one or more LEDs of the PPG sensor to be set to some value based on the skin tone value. In another example, the processing circuitry of the wearable device causes a wavelength of the LEDs to take a value based on the skin tone value (e.g., green, yellow, etc.), i.e. the parameter of the PPG sensor measurement based on the skin tone value is the wavelength of the one or more LEDs of the PPG sensor. Further, both the optical power and the wavelength may be set depending on the skin tone data. Note that in the following the term “calibration” may also cover adjusting a parameter of the PPG sensor measurement based on the skin tone data (without necessarily carrying out a calibration measurement and comparing the results of the calibration measurement to pre-determined values). The calibration of the PPG sensor is performed, in some implementations, using a lookup table or a machine learning engine. Note that “obtaining” skin tone data may comprise obtaining the skin tone data indirectly, e.g., by receiving an image from which the skin tone data can be derived. In other words, the method may also (in addition or as an alternative to directly receiving skin tone data) relate to receiving, by the wearable device and from the mobile device, an image and deriving skin tone data from the received image.

In one general aspect, a method includes receiving, by a wearable device worn by a user, the wearable device including a photoplethysmography (PPG) sensor, an indication that a measurement with the PPG sensor is to be performed. The method also includes obtaining, from a mobile device connected to the wearable device, skin tone data indicating a skin tone of the user. The method further includes adjusting a parameter of the PPG sensor based on the skin tone data.

In another general aspect, a computer program product includes a nontransitory storage medium, the computer program product including code that, when executed by at least one processor, causes the at least one processor to perform a method. The method includes receiving, by a wearable device worn by a user, the wearable device including a photoplethysmography (PPG) sensor, an indication that a measurement with the PPG sensor is to be performed. The method also includes obtaining, from a mobile device connected to the wearable device, skin tone data indicating a skin tone of the user. The method further includes adjusting a power output of a set of light-emitting diodes (LEDs) of the PPG sensor based on the skin tone data, the set of LEDs being configured to transmit electromagnetic radiation to skin of the user.

In another general aspect, a wearable device worn by a user can include memory and processing circuitry coupled to the memory. The processing circuitry is configured to receive an indication that a measurement with the PPG sensor is to be performed. The processing circuitry is also configured to obtain, from a mobile device connected to the wearable device, skin tone data indicating a skin tone of the user. The processing circuitry is further configured to adjust a wavelength of a set of light-emitting diodes (LEDs) of the PPG sensor based on the skin tone data, the set of LEDs being configured to transmit electromagnetic radiation to skin of the user.

The details of one or more implementations are set forth in the accompanying drawings and the description below. Other features will be apparent from the description and drawings, and from the claims.

### BRIEF DESCRIPTION OF THE DRAWINGS

FIG. 1A is a diagram that illustrates an example wearable device, in accordance with implementations described herein.

FIG. 1B is a diagram that illustrates a rear view of the example wearable device, in accordance with implementations described herein.

FIG. 1C is a diagram that illustrates a usage of the example wearable device.

FIG. 2 is a sequence diagram that illustrates an example interaction between a wearable device and a mobile device.

FIG. 3 is a diagram that illustrates an example electronic environment for calibrating a PPG sensor using skin tone data obtained from images on a mobile device.

FIG. 4 is a flow chart that illustrates an example method of calibrating a PPG sensor using skin tone data obtained from images on a mobile device.

### DETAILED DESCRIPTION

This disclosure relates to photoplethysmography (PPG). PPG uses electromagnetic (EM) radiation (usually in the infrared (IR) band) to measure volumetric variations of blood circulation. A typical PPG device contains a source of EM radiation and a photodetector. The EM radiation source emits EM radiation to a tissue and the photodetector measures the reflected light from the tissue. The reflected light is proportional to blood volume variations. Many PPG sensors use an infrared light emitting diode (IR-LED) or a green LED as the main EM radiation source. IR-LEDs may be used for measuring the flow of blood that is more deeply concentrated in certain parts of body such as the muscles, whereas green light may be used for calculating the absorption of oxygen in oxyhemoglobin (oxygenated blood) and deoxyhemoglobin (blood without oxygen present). For example, a PPG device may be used to perform pulse oximetry, e.g., measure oxygen level in blood.

Wearable PPG sensors can be placed at certain body locations, including the wrist. Such sensors may be inexpensive, highly portable, and very convenient to wear by its users. Many PPG sensors emit light at the tissue site with one or more LEDs. The photodiode measures the intensity of the non-absorbed light reflected from the tissue. The LED colors used include red and green; however, in some cases a yellow LED has also been used. Light with longer wavelengths penetrates more deeply into the tissue.

A technical problem with the above-described wearable PPG sensors is that the measurements made by PPG sensors can be sensitive to skin tone at the wrist. Accordingly, the PPG sensors may not provide accurate measurements without calibrating light brightness or wavelength to skin tone.

A technical solution to the technical problem includes accessing photos on a mobile device connected to a wearable device having PPG sensors and deriving skin tone information from the photos. Based on the skin tone information, the wearable device sets a parameter value (e.g., power, wavelength) of the PPG sensor (e.g., when performing a calibration operation on the PPG sensor). For example, upon a first PPG measurement from a user, processing circuitry of the wearable device determines that there is a connection between the wearable device and a mobile device belonging to the user. Once the determination is made, the wearable device sends the mobile device a prompt to select at least one image (photo) of the user. Each photo, in some implementations, has metadata that indicates a value for the skin tone of the user. In one example, the value of the skin tone is a number between 1 and 6 inclusive (e.g., the Fitzpatrick Skin Phototype Classification). In another example, the value of the skin tone is an alphanumeric identifier. The mobile device sends the skin tone value to the wearable device. The processing circuitry of the wearable device then performs a calibration operation on the PPG sensor based on the skin tone value. In one example, the processing circuitry of the wearable device causes an optical power of the LEDs of the PPG sensor to be set to some value based on the skin tone value. In another example, the processing circuitry of the wearable device causes a wavelength of the LEDs to take a value based on the skin tone value (e.g., green, yellow, etc.). The calibration of the PPG sensor is performed, in some implementations, using a lookup table or a machine learning engine.

A technical advantage of the above-described technical solution is that the technical solution is convenient to the user and ensures that the measurements of the PPG sensor are accurate.

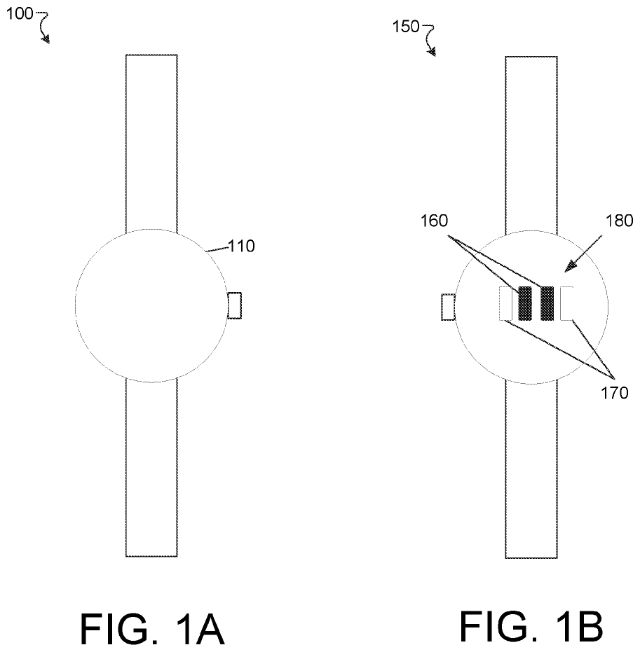

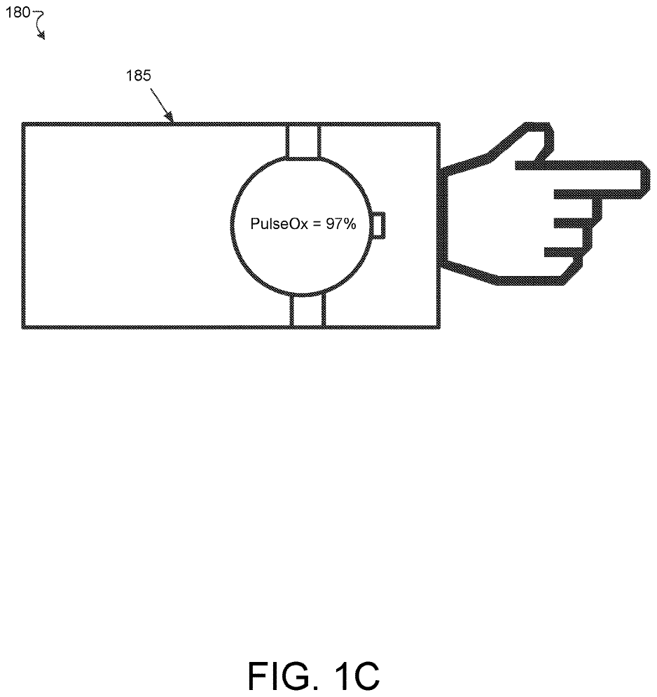

FIG. 1A is a diagram that illustrates a top-down view of an example wearable device 100. As shown in FIG. 1A, the example wearable device 100 is a smartwatch. In some implementations, the wearable device 100 is a wristband. The wearable device 100 has processing circuitry 110 which is used to perform various functions as described herein.

FIG. 1B is a diagram that illustrates a rear view 150 of the wearable device 100. As shown in FIG. 1B, the wearable device 100 includes a set of LEDs 160 and light detectors 170 of a PPG sensor 180. The set of LEDs 160 is configured to produce and transmit light of a specified wavelength and power to the skin of the user's wrist. The set of detectors 170 is configured to detect reflected light power at the specified wavelength from the user's skin.

The processing circuitry 110 is configured to perform a calibration operation on the PPG sensor 180. In performing the calibration operation, the processing circuitry 110 receives at least one image (photo) from a mobile device connected to the wearable device 100. The processing circuitry 110 then determines, based on the at least one image, a skin tone value for the user. In some implementations, the processing circuitry 110 reads metadata of the at least one image that provides a skin tone value. It is also possible that the processing circuitry 110 receives the skin tone data from the mobile device. In some implementations, the processing circuitry 110 or a corresponding processing circuitry of the mobile device inputs the at least one image into a machine learning engine configured to determine the skin tone value from the at least one image. Once the processing circuitry 110 determines the skin tone value of the user, the processing circuitry 110 adjusts one or both of a transmission power of the set of LEDs 160 or a wavelength of the electromagnetic radiation emitted by the set of LEDs 160. In some implementations, the wavelength being adjusted is a mean wavelength of the set of LEDs 160. In some implementations, the wavelength being adjusted is a peak wavelength of the set of LEDs 160. For example, the wavelength of the LEDs 160 may be changed from that corresponding to yellow (about 550 nm) to that corresponding to green (about 500 nm). In some implementations, the set of detectors 170 are adjusted based on the change of wavelength in the set of LEDs 160. In some implementations, the change in transmission power or the change in wavelength is determined by the processing circuitry 110 based on a lookup table. In some implementations, the change in transmission power or the change in wavelength is determined by the processing circuitry 110 based on results of a machine learning engine that takes in the at least one image of the user as input. The processing performed by the processing circuitry 110 described above may also be carried out by a processing circuitry of the mobile device.

FIG. 1C is a diagram that illustrates an example usage 180 of the wearable device 100 as worn on a wrist 185. As shown in FIG. 1C, the PPG sensor built into the wearable device 100 may perform pulse oximetry. That is, the watch face may display the result of the pulse oximetry based on measurements made by the PPG sensor's LEDs and light detectors.

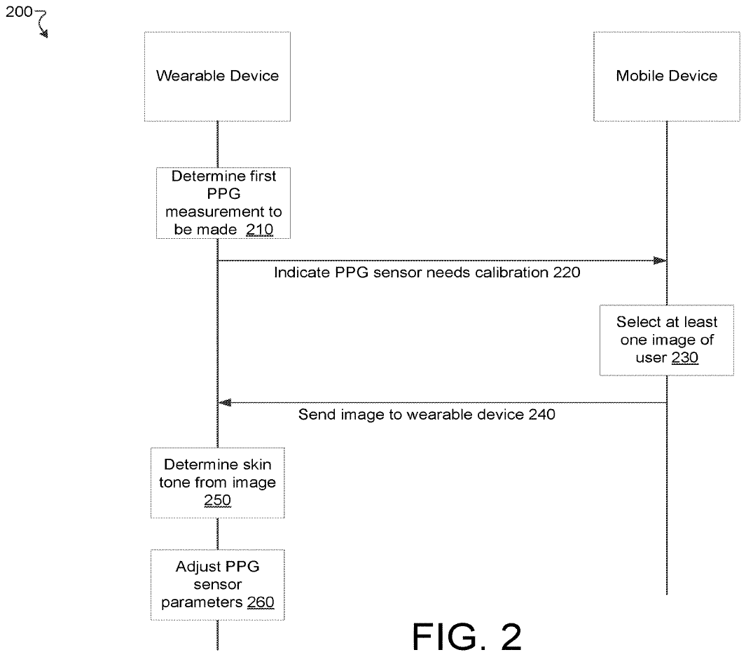

FIG. 2 is a sequence diagram that illustrates an example interaction 200 between a wearable device (e.g., wearable device 100) and a mobile device. In some implementations, the wearable device and mobile device are paired over a wireless connection. In some implementations, the wireless connection is a Bluetooth connection.

At 210, the wearable device determines that a first PPG measurement needs to be made, and that its PPG sensor needs to be calibrated. In one example, the wearable device may be new and just taken from its box for the first time. In another example, a new user registers the wearable device as their own for the first time. In some implementations, the PPG sensor is configured to make measurements (e.g., for pulse oximetry) automatically and/or continuously. In some implementations, the PPG sensor makes the measurements on demand by the user.

At 220, the wearable device sends the mobile device a message indicating that the PPG sensor needs a calibration. In some implementations, the indication includes a request for photos of the user.

At 230, the mobile device performs a selection of at least one image of the user. In some implementations, the mobile device provides a prompt for the user to select the at least one image stored on the mobile device or in a cloud server. In some implementations, the mobile device automatically selects the least one image of the user. In some implementations, the automatic selection of the at least one image of the user is based on metadata of the at least one image identifying the at least one image of the user as an image of the user. In some implementations, such metadata includes a skin tone value.

At 240, the mobile device sends the at least one image of the user to the wearable device.

At 250, the wearable device determines the skin tone value from the at least one image of the user. In some implementations, the skin tone value is included in metadata of the at least one image. In such implementations, processing circuitry of the wearable device (e.g., processing circuitry 110) determines whether there is a skin tone value identifier in the metadata (e.g., “STV”) and, if so, stores the skin tone value associated with the identifier. In some implementations, the skin tone value is a counting number between 1 and 6. In some implementations, the skin tone value is a real number. In some implementations, there is no skin tone value in the metadata of the at least one image. In some implementations, the processing circuitry inputs the at least one image into a machine learning engine (e.g., a convolutional neural network) that is configured to determine a skin tone value based on at least one image. As set out above, the skin tone data may also be determined by the mobile device (using the image and similarly as in step 250). According to this embodiment, the wearable device only receives the skin tone data from the mobile device.

At 260, the wearable device performs a calibration operation on the PPG sensor, e.g., the processing circuitry adjusts PPG sensor parameters based on the skin tone value. In some implementations, the calibration operation includes adjusting a power of the transmitted electromagnetic radiation (light) produced by the LEDs of the PPG sensor. For example, for darker skin tones, the processing circuitry may increase a power of the transmitted light to compensate for higher absorption of the light in the skin. For lighter skin tones, the processing circuitry may decrease a power of the transmitted light to compensate for lower absorption of the light in the skin. In some implementations, the calibration operation includes adjusting a wavelength of the transmitted electromagnetic radiation (light) produced by the LEDs of the PPG sensor. It is also possible that the mobile device determines the skin tone data, determines the parameter of the PPG sensor measurement (e.g., the light power and/or the wavelength) and transmits the parameter to the wearable device.

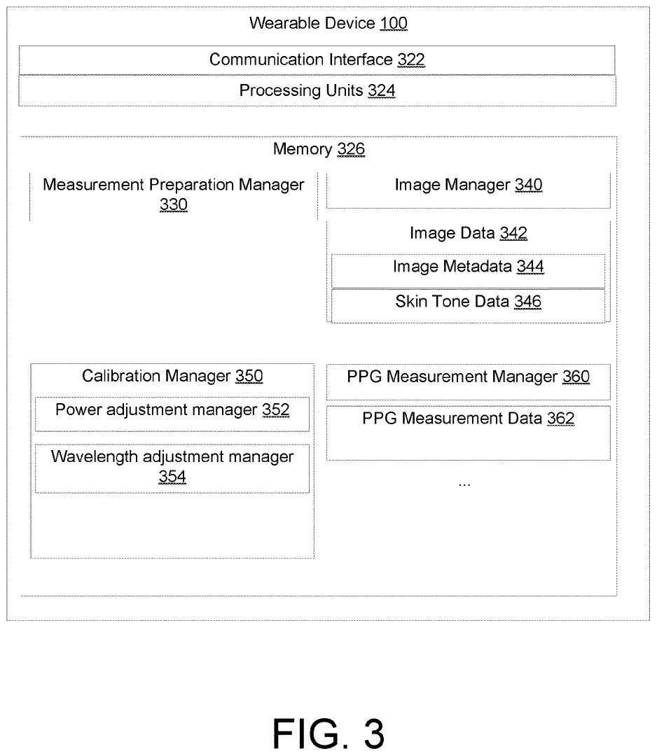

FIG. 3 is a diagram that illustrates an example electronic environment for performing a calibration operation of a PPG sensor in the wearable device 100. The wearable device 100 includes a communication interface 322, one or more processing units 324, and nontransitory memory 326. The communication interface 322 includes, for example, Bluetooth adaptors, and the like, for converting electronic and/or optical signals received from the network to electronic form for use by the wearable device 330. The set of processing units 324 include one or more processing chips and/or assemblies. The memory 326 includes both volatile memory (e.g., RAM) and non-volatile memory, such as one or more ROMs, disk drives, solid state drives, and the like. The set of processing units 324 and the memory 326 together form processing circuitry, which is configured and arranged to carry out various methods and functions as described herein.

In some implementations, one or more of the components of the wearable device 100 can be, or can include processors (e.g., processing units 324) configured to process instructions stored in the memory 326. Examples of such instructions as depicted in FIG. 3 include measurement preparation manager 330, image manager 340, calibration manager 350, and PPG measurement manager 360. Further, as illustrated in FIG. 3, the memory 326 is configured to store various data, which is described with respect to the respective managers that use such data.

The measurement preparation manager 330 is configured to determine that a calibration needs to be performed on the PPG sensor (e.g., PPG sensor 180). In some implementations, such a determination is made when a new user registers the wearable device 100 (e.g., identifies themselves as a new user to the wearable device). If no such determination is made, no calibration is performed on the PPG sensor.

The image manager 340 is configured to receive at least one image (image data 342) from a mobile device (not shown) and determine a value of the skin tone of the user based on the at least one image. In some implementations, the image data 342 includes image metadata 344 which includes a skin tone value (skin tone data 346). In some implementations, the image manager 340 determines the skin tone data 346 based on inputting the image data 342 into a machine learning engine configured to output a skin tone value based on at least one image.

The calibration manager 350 is configured to perform a calibration operation on the PPG sensor of the wearable device 100 based on the skin tone data 346. As shown in FIG. 3, the calibration manager 350 includes a power adjustment manager 352 and a wavelength adjustment manager 354. The calibration manager 350, by performing the calibration operation, determines whether the power adjustment manager 352 or the wavelength adjustment manager 354 (or both) is used in the calibration of the PPG sensor.

The power adjustment manager 352 is configured to adjust a power of transmitted electromagnetic radiation (e.g., light) emitted from a set of LEDs of the PPG sensor. For example, for darker skin tones, the power adjustment manager 352 may increase a power of the transmitted light to compensate for higher absorption of the light in the skin. For lighter skin tones, the power adjustment manager 352 may decrease a power of the transmitted light to compensate for lower absorption of the light in the skin.

The wavelength adjustment manager 354 is configured to adjust a wavelength of the emitted light from the set of LEDs of the PPG sensor. In some implementations, the wavelength adjustment manager 354 is also configured to perform an adjustment on the detector(s) (i.e., set of detectors 170) of the PPG sensor commensurate with the adjustment made to the wavelength of the emitted light.

The PPG measurement manager 360 is configured to perform a measurement using the PPG sensor and acquire PPG measurement data 362 as a result.

The components (e.g., modules, processing units 324) of wearable device 100 can be configured to operate based on one or more platforms (e.g., one or more similar or different platforms) that can include one or more types of hardware, software, firmware, operating systems, runtime libraries, and/or so forth. In some implementations, the components of the wearable device 100 can be configured to operate within a cluster of devices (e.g., a server farm). In such an implementation, the functionality and processing of the components of the wearable device 100 can be distributed to several devices of the cluster of devices.

The components of the wearable device 100 can be, or can include, any type of hardware and/or software configured to perform calibration operations on a PPG sensor. In some implementations, one or more portions of the components shown in the components of the wearable device 100 in FIG. 3 can be, or can include, a hardware-based module (e.g., a digital signal processor (DSP), a field programmable gate array (FPGA), a memory), a firmware module, and/or a software-based module (e.g., a module of computer code, a set of computer-readable instructions that can be executed at a computer). For example, in some implementations, one or more portions of the components of the wearable device 100 can be, or can include, a software module configured for execution by at least one processor (not shown). In some implementations, the functionality of the components can be included in different modules and/or different components than those shown in FIG. 3, including combining functionality illustrated as two components into a single component.

Although not shown, in some implementations, the components of the wearable device 100 (or portions thereof) can be configured to operate within, for example, a data center (e.g., a cloud computing environment), a computer system, one or more server/host devices, and/or so forth. In some implementations, the components of the wearable device 100 (or portions thereof) can be configured to operate within a network. Thus, the components of the wearable device 100 (or portions thereof) can be configured to function within various types of network environments that can include one or more devices and/or one or more server devices. For example, the network can be, or can include, a local area network (LAN), a wide area network (WAN), and/or so forth. The network can be, or can include, a wireless network and/or wireless network implemented using, for example, gateway devices, bridges, switches, and/or so forth. The network can include one or more segments and/or can have portions based on various protocols such as Internet Protocol (IP) and/or a proprietary protocol. The network can include at least a portion of the Internet.

In some implementations, one or more of the components of the wearable device 100 can be, or can include, processors configured to process instructions stored in a memory. For example, measurement preparation manager 330 (and/or a portion thereof), image manager 340 (and/or a portion thereof), calibration manager 350 (and/or a portion thereof), and PPG measurement manager 460 (and/or a portion thereof) are examples of such instructions.

In some implementations, the memory 326 can be any type of memory such as a random-access memory, a disk drive memory, flash memory, and/or so forth. In some implementations, the memory 326 can be implemented as more than one memory component (e.g., more than one RAM component or disk drive memory) associated with the components of the wearable device 100. In some implementations, the memory 326 can be a database memory. In some implementations, the memory 326 can be, or can include, a non-local memory. For example, the memory 326 can be, or can include, a memory shared by multiple devices (not shown). In some implementations, the memory 326 can be associated with a server device (not shown) within a network and configured to serve the components of the wearable device 100. As illustrated in FIG. 3, the memory 326 is configured to store various data, including image data 342 and PPG measurement data 362.

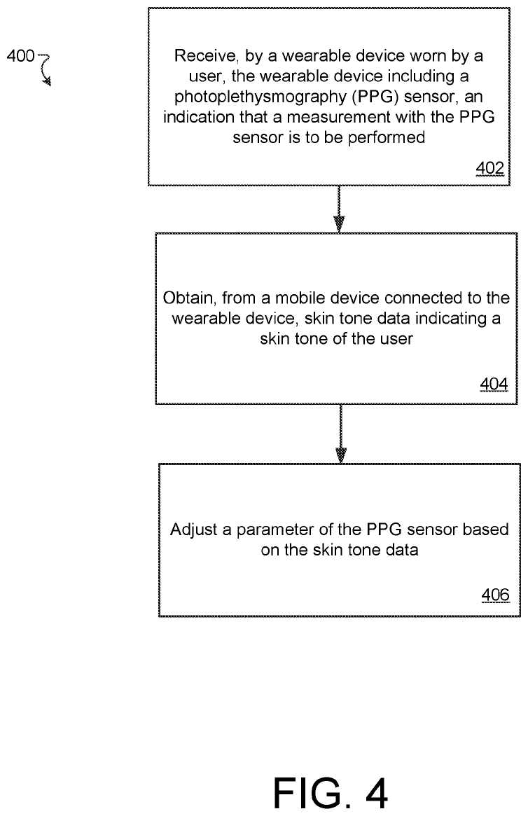

FIG. 4 is a flow chart that illustrates an example method 400 of performing a calibration operation on a PPG sensor. The method 400 may be performed using the wearable device 100 of FIG. 3.

At 402, a measurement preparation manager (e.g., measurement preparation manager 330) receives an indication that a measurement with a PPG sensor is to be performed.

At 404, an image manager (e.g., image manager 340) obtains, from a mobile device connected to the wearable device 100, skin tone data (e.g., skin tone data 346) indicating a skin tone of the user.

At 406, a calibration manager (e.g., calibration manager 350) performs a calibration operation on the PPG sensor based on the skin tone data.

Specific structural and functional details disclosed herein are merely representative for purposes of describing example embodiments. Example embodiments, however, may be embodied in many alternate forms and should not be construed as limited to only the embodiments set forth herein.

The terminology used herein is for the purpose of describing particular embodiments only and is not intended to be limiting of the embodiments. As used herein, the singular forms “a,” “an,” and “the” are intended to include the plural forms as well, unless the context clearly indicates otherwise. It will be further understood that the terms “comprises,” “comprising,” “includes,” and/or “including,” when used in this specification, specify the presence of the stated features, steps, operations, elements, and/or components, but do not preclude the presence or addition of one or more other features, steps, operations, elements, components, and/or groups thereof.

It will be understood that when an element is referred to as being “coupled,” “connected,” or “responsive” to, or “on,” another element, it can be directly coupled, connected, or responsive to, or on, the other element, or intervening elements may also be present. In contrast, when an element is referred to as being “directly coupled,” “directly connected,” or “directly responsive” to, or “directly on,” another element, there are no intervening elements present. As used herein the term “and/or” includes any and all combinations of one or more of the associated listed items.

Spatially relative terms, such as “beneath,” “below,” “lower,” “above,” “upper,” and the like, may be used herein for ease of description to describe one element or feature in relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 70 degrees or at other orientations) and the spatially relative descriptors used herein may be interpreted accordingly.

Example embodiments of the concepts are described herein with reference to cross-sectional illustrations that are schematic illustrations of idealized embodiments (and intermediate structures) of example embodiments. As such, variations from the shapes of the illustrations as a result, for example, of manufacturing techniques and/or tolerances, are to be expected. Thus, example embodiments of the described concepts should not be construed as limited to the particular shapes of regions illustrated herein but are to include deviations in shapes that result, for example, from manufacturing. Accordingly, the regions illustrated in the figures are schematic in nature and their shapes are not intended to illustrate the actual shape of a region of a device and are not intended to limit the scope of example embodiments.

It will be understood that although the terms “first,” “second,” etc. may be used herein to describe various elements, these elements should not be limited by these terms. These terms are only used to distinguish one element from another. Thus, a “first” element could be termed a “second” element without departing from the teachings of the present embodiments.

Unless otherwise defined, the terms (including technical and scientific terms) used herein have the same meaning as commonly understood by one of ordinary skill in the art to which these concepts belong. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

While certain features of the described implementations have been illustrated as described herein, many modifications, substitutions, changes, and equivalents will now occur to those skilled in the art. It is, therefore, to be understood that the appended claims are intended to cover such modifications and changes as fall within the scope of the implementations. It should be understood that they have been presented by way of example only, not limitation, and various changes in form and details may be made. Any portion of the apparatus and/or methods described herein may be combined in any combination, except mutually exclusive combinations. The implementations described herein can include various combinations and/or sub-combinations of the functions, components, and/or features of the different implementations described.

1. A method, comprising:
receiving, by a wearable device worn by a user, the wearable device including a photoplethysmography (PPG) sensor, an indication that a measurement with the PPG sensor is to be performed;
obtaining, from a mobile device connected to the wearable device, skin tone data indicating a skin tone of the user; and
adjusting a parameter of the PPG sensor based on the skin tone data.

2. The method as in claim 1, wherein obtaining the skin tone data includes: receiving at least one image of the user.

3. The method as in claim 2, wherein obtaining the skin tone data further includes:
acquiring, from the at least one image of the user, image metadata indicating the skin tone of the user.

4. The method as in claim 1, wherein the wearable device is worn on a wrist of the user.

5. The method as in claim 1, wherein the wearable device and the mobile device are connected via a Bluetooth connection.

6. The method as in claim 1, wherein adjusting the parameter of the PPG sensor includes:
adjusting a power output of one or more light-emitting diodes (LEDs) of the PPG sensor, the one or more LEDs being configured to transmit electromagnetic radiation to skin of the user.

7. The method as in claim 1, wherein adjusting the parameter of the PPG sensor includes:
adjusting a wavelength of one or more light-emitting diodes (LEDs) of the PPG sensor, the one or more LEDs being configured to transmit electromagnetic radiation to skin of the user.

8. A computer program product comprising a nontransitory storage medium, the computer program product including code that, when executed by at least one processor, causes the at least one processor to perform a method as in claim 1.

9. A computer program product comprising a nontransitory storage medium, the computer program product including code that, when executed by at least one processor, causes the at least one processor to perform a method, the method comprising:
receiving, by a wearable device worn by a user, the wearable device including a photoplethysmography (PPG) sensor, an indication that a measurement with the PPG sensor is to be performed;
obtaining, from a mobile device connected to the wearable device, skin tone data indicating a skin tone of the user; and
adjusting a power output of a set of light-emitting diodes (LEDs) of the PPG sensor based on the skin tone data, the set of LEDs being configured to transmit electromagnetic radiation to skin of the user and/or adjusting a wavelength of the set of LEDs of the PPG sensor.

10. The computer program product as in claim 9, wherein obtaining the skin tone data includes:
receiving at least one image of the user.

11. The computer program product as in claim 9, wherein obtaining the skin tone data further includes:
acquiring, from the at least one image of the user, image metadata indicating the skin tone of the user.

12. The computer program product as in claim 9, wherein the wearable device is worn on a wrist of the user.

13. The computer program product as in claim 9, wherein the wearable device and the mobile device are connected via a Bluetooth connection.

14. A wearable device worn by a user, the wearable device including a photoplethysmography (PPG) sensor, comprising:
memory; and
processing circuitry coupled to the memory, the processing circuitry being configured to perform a method as in any of claim 1.

15. A wearable device worn by a user, the wearable device including a photoplethysmography (PPG) sensor, comprising:
memory; and
processing circuitry coupled to the memory, the processing circuitry being configured to:
receive an indication that a measurement with the PPG sensor is to be performed;
obtain, from a mobile device connected to the wearable device, skin tone data indicating a skin tone of the user; and
adjust a wavelength of a set of light-emitting diodes (LEDs) of the PPG sensor based on the skin tone data, the set of LEDs being configured to transmit electromagnetic radiation to skin of the user and/or adjust a power output of the set of light-emitting diodes (LEDs) of the PPG sensor.

16. The wearable device as in claim 15, wherein the processing circuitry configured to obtain the skin tone data is further configured to:
receive at least one image of the user.

17. The wearable device as in claim 15, wherein the processing circuitry configured to obtain the skin tone data is further configured to:
acquire, from the at least one image of the user, image metadata indicating the skin tone of the user.

18. The wearable device as in claim 15, wherein the wearable device is worn on a wrist of the user.

19. The wearable device as in claim 15, wherein the wearable device and the mobile device are connected via a Bluetooth connection.
